(19) United States
(12) Patent Application Publication
(10) Pub. No.: US 2024/0010775 A1
(43) Pub. Date: Jan. 11, 2024

(54) COPOLYMER HAVING VISCOELASTIC AND SUSPENSIVE PROPERTIES

(71) Applicant: COATEX, Genay (FR)

(72) Inventors: Laurie PARRENIN, Villeneuve (FR); Jean-Marc SUAU, Lucenay (FR)

(73) Assignee: COATEX, Genay (FR)

(21) Appl. No.: 18/253,992

(22) PCT Filed: Dec. 7, 2021
(86) PCT No.: PCT/FR2021/000143

§ 371 Date: May 23, 2023

(30) Foreign Application Priority Data
Dec. 9, 2020 (FR) FR20 12936

(51) Int. Cl. C08F 220/06; C08F 220/10; C08K 5/42

(52) U.S. Cl. CPC C08F 220/06; C08F 220/10; C08K 5/42

(57) ABSTRACT

A crosslinked, sulphonated, acrylic copolymer may be prepared from at least four monomers and in the absence of methacrylic acid. Such a copolymer may be prepared into an aqueous composition having viscoelastic and suspensive properties, including such a copolymer. Such copolymer(s) may include, in polymerized form: (a) an anionic monomer comprising acrylic acid and/or an acrylic acid oligomer, optionally in salt form; (b) a non-ionic monomer comprising styrene, a C1-C8 acrylate ester, and/or a C1-C8 methacrylate ester; (c) a compound comprising 2-acrylamido-2-methylpropane sulfonic acid, 2-sulfoethyl methacrylate, sodium methallyl sulfonate, and/or styrene sulfonate, optionally in salt form; (d) a cross-linking compound or compound including a first and a second polymerizable olefinic unsaturation, wherein the copolymer comprises no methacrylic acid

The invention provides a cross-linked, sulphonated acrylic copolymer P prepared from at least four monomers and in the absence of methacrylic acid. The invention also comprises the preparation of an aqueous composition with viscoelastic and suspensive properties and comprising this copolymer P.

Many technical fields require the use of agents that combine several properties. In particular, some technical fields require control over the rheology and texture of a composition. The agents used for this purpose must also make it possible to stabilise these compositions.

In the field of coating compositions, in particular for wood stain compositions, the agents used must make it possible to control the rheology but also to confer a suspensive effect on these compositions that comprise particles. Wood stain compositions usually combine a solvent, in particular water, with a binder, for example an acrylic binder, an alkyd binder or an alkyd-urethane binder, and pigment particles. These compositions make it possible to protect the substrate on which they are applied, particularly a wood or concrete substrate. The compatibility of the different constituents of a composition is therefore also essential. In particular, the rheology and texture control agents of a composition must have a glass transition temperature that makes it possible to effectively combine them with other substances present in these compositions, in particular with a binder, generally in the form of particles.

The desired suspensive effect of a composition is the ability to keep particles in suspension in a continuous phase, in a stable manner over time, for example while the composition is stored. The particles are generally solid bodies, hollow or solid. They may also be liquid entities that cannot be mixed with the continuous phase or be encapsulated or in gaseous form. Their shape, texture and structure can vary widely, in particular depending on the expected final properties.

Suspensive performances can be assessed by determining the value of the elastic modulus G′, the value of the damping factor (Tan δ) and the value of the elastic strength.

Moreover, it is necessary to control the viscosity of these compositions, both for low or medium shear gradients and for high shear gradients. Indeed, during its preparation, storage, application or drying, a composition may be subjected to numerous stresses requiring particularly complex rheological properties, in particular viscoelastic properties. In rheology, the behaviour of a straight viscoelastic material is intermediate between that of an ideal elastic solid symbolised by a spring of modulus E (or G) and that of a Newtonian viscous liquid symbolised by a viscosity damping factor. The elasticity of a material reflects its ability to retain and return energy after deformation. The viscosity of a material reflects its ability to dissipate energy.

The viscoelasticity of a composition must therefore also be improved so that this composition has both viscous and elastic characteristics when it is deformed. The viscous component allows this composition to withstand shear flow and exhibit deformation that increases linearly over time when stress is applied. The elastic component allows a deformation when stress is applied and then the return to the original state once the stress is suspended.

There is therefore a need to have agents that can provide viscoelastic and suspensive properties to compositions.

Moreover, and particularly for environmental reasons, there is also a great need for compositions comprising no methacrylic acid, while offering sustained or improved performance compared to the compositions in the prior art. Indeed, the use of methacrylic acid, particularly methacrylic acid prepared from acetone cyanohydrin, which is a highly toxic compound, should be limited as far as possible.

Documents US2017003717 and US20190315897 describe copolymers prepared with methacrylic acid. Document US20080193405 describes the preparation of hydraulic or alcoholic compositions comprising a combination of copolymers associated with a mixture of acrylic acid and of a cross-linking compound. Document WO2014185381 relates to a binder composition for a lithium battery and which comprises a fluorinated copolymer.

There is therefore a need to have a copolymer with improved properties and that makes it possible to provide a solution to all or part of the problems of the polymers of the prior art.

Thus, the invention provides a copolymer P prepared in the absence of methacrylic acid, by at least one polymerisation reaction:
a. of at least one anionic monomer (a) chosen among acrylic acid, an acrylic acid salt, acrylic acid oligomers, acrylic acid oligomer salts and combinations thereof;
b. of at least one non-ionic monomer (b) chosen among styrene, C₁-C₈ esters of an acid chosen among acrylic acid, methacrylic acid and combinations thereof:
c. of at least one compound (c) chosen among 2-acrylamido-2-methylpropane sulphonic acid, 2-sulphoethyl methacrylate, sodium methallyl sulphonate, styrene sulphonate, their salts and combinations thereof;
d. of at least one cross-linking compound (d) or comprising at least two polymerisable olefinic unsaturations.

Preferably according to the invention, copolymer P can be prepared from compounds a, b, c and d alone.

Also preferably according to the invention, monomer (a) is acrylic acid or a mixture of acrylic acid oligomers of formula (I):

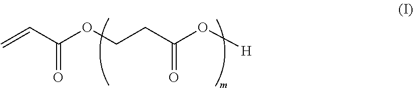

wherein m is an integer or decimal ranging from 1 to 10, preferably ranging from 2 to 4. Preferably according to the invention, monomer (a) is acrylic acid.

Also preferably according to the invention, monomer (b) is chosen among:
alkyl acrylate, in particular C₁-C₈-alkyl acrylate, preferentially C₁-C₄-alkyl acrylate, more preferentially methyl acrylate, ethyl acrylate, propyl acrylate, isobutyl acrylate, n-butyl acrylate,
alkyl methacrylate, in particular C₁-C₈-alkyl methacrylate, preferentially C₁-C₄-alkyl methacrylate, more preferentially methyl methacrylate, ethyl methacrylate, propyl methacrylate, isobutyl methacrylate, n-butyl methacrylate,
aryl acrylate, preferably phenyl acrylate, benzyl acrylate, phenoxy ethyl acrylate,
aryl methacrylate, preferably phenyl methacrylate, benzyl methacrylate, phenoxy ethyl methacrylate and
combinations thereof.

More preferably according to the invention, monomer (b) is chosen among ethyl acrylate and butyl acrylate, more preferentially ethyl acrylate.

Preferably, monomer (b) according to the invention is a non-fluorinated monomer or monomer (b) is different from 2,2,2-trifluoroethyl methacrylate. According to the invention, the preferred monomer (c) is chosen among 2-acrylamido-2-methylpropane sulphonic acid (AMPS) and its sodium or ammonium salts. According to the invention, the preferred compound (d) is chosen among the polyunsaturated aromatic monomers such as divinyl benzene, divinyl naphthalene and trivinyl benzene, the polyunsaturated alicyclic monomers such as 1,2,4-trivinylcyclohexane, divalent phthalic acid esters such as diallyl phthalate, polyalkenyl ethers such as triallyl pentaerythritol, diallyl pentaerythritol, diallyl sucrose, octaallyl sucrose and trimethylol propane diallyl ether, polyunsaturated polyalcohol or polyacid esters such as 1,6-hexanediol di(meth)acrylate, tetramethylene tri(meth)acrylate, allyl acrylate, diallyl itaconate, diallyl fumarate, diallyl maleate, trimethylol propane tri(meth)acrylate, trimethylol propane di(meth)acrylate, polyalkylene oxyglycol di(meth)acrylate and polyethylene glycol di(meth)acrylate, alkylene bisacryl amides such as methylene bisacryl amide and propylene bisacryl amide, hydroxy or carboxy derivatives of methylene bis-acrylamide such as N,N′-bismethylol methylene bisacrylamide, polyalkylene glycol di(meth)acrylates such as ethylene glycol di(meth)acrylate, diethylene glycol di(meth)acrylate, triethylene glycol di(meth)acrylate, allyl methacrylate, ethylene glycol dimethacrylate, butylene glycol dimethacrylate, pentaerythritol di-, tri- and tetra-acrylates, polyalkylene oxyglycol di(meth)acrylates such as polyethylene glycol diacrylate, bisphenol A diacrylate, butane diol dimethacrylate, 2,2-dimethylpropanediol dimethacrylate, phenylene diacrylate, an asymmetric cross-linking compound and mixtures thereof.

The preferred asymmetric cross-linking compound (d) according to the invention is chosen among:
a compound of formula (II):

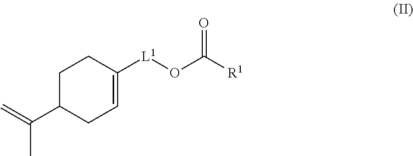

wherein:
   L¹ represents CH₂, CH₂ monoalkoxylate or CH₂ polyalkoxylate,
   R¹ represents —C(H)═CH₂, —C(CH₃)═CH₂, —C(H)═C(H)C(O)OH, —C(H)═C(H)CH₃, —C(═CH₂)CH₂C(O)OH, —CH₂C(═CH₂)C(O)OH, QiOQ²OC(O)C(CH₃)═CH₂ or QiOQ²OC(O)C(H)═CH₂,
   Q¹ represents a divalent residue of an asymmetric diisocyanate compound, preferably chosen among tolyl-1,3-diisocyanate (TDI) and isophorone-diisocyanate (IPDI),
   Q² represents CH₂, CH₂—CH₂, CH₂ monoalkoxylate, CH₂—CH₂ monoalkoxylate, CH₂ polyalkoxylate or CH₂—CH₂ polyalkoxylate; preferably a compound of formula (II) wherein:
   L¹ represents CH₂ and
   R¹ represents —C(H)═CH₂, —C(CH₃)═CH₂, —C(H)═C(H)C(O)OH, —C(H)═C(H)CH₃, —C(═CH₂)CH₂C(O)OH, —CH₂C(═CH₂)C(O)OH or a compound of formula (II) wherein:
   L¹ represents CH₂ monoalkoxylate or CH₂ polyalkoxylate,
   R¹ represents QiOQ²OC(O)C(CH₃)═CH₂ or QiOQ²OC(O)C(H)═CH₂,
   Q¹ represents a divalent residue of an asymmetric diisocyanate compound, preferably chosen among tolyl-1,3-diisocyanate (TDI) and isophorone-diisocyanate (IPDI),
   Q² represents CH₂, CH₂—CH₂, CH₂ monoalkoxylate, CH₂—CH₂ monoalkoxylate, CH₂ polyalkoxylate or CH₂—CH₂ polyalkoxylate;
a compound of formula (III):

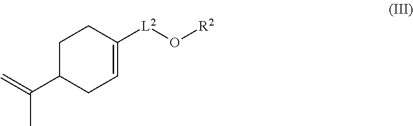

wherein:
   L² represents CH₂, CH₂ monoalkoxylate or CH₂ polyalkoxylate,
   R² represents —C(H)═CH₂, —C(CH₃)═CH₂, —C(H)═C(H)C(O)OH, —C(H)═C(H)CH₃, —C(═CH₂)CH₂C(O)OH, —CH₂C(═CH₂)C(O)OH, Q³OQ⁴OC(O)C(CH₃)═CH₂ or Q³OQ⁴OC(O)C(H)═CH₂,
   Q³ represents a divalent residue of an asymmetric diisocyanate compound, preferably chosen among tolyl-1,3-diisocyanate (TDI) and isophorone diisocyanate (IPDI),
   Q⁴ represents CH₂, CH₂—CH₂, CH₂ monoalkoxylate, CH₂—CH₂ monoalkoxylate, CH₂ polyalkoxylate or CH₂—CH₂ polyalkoxylate; preferably a compound of formula (III) wherein:
   L² represents CH₂,
   R² represents —C(H)═CH₂, —C(CH₃)═CH₂, —C(H)═C(H)C(O)OH, —C(H)═C(H)CH₃, —C(═CH₂)CH₂C(O)OH, —CH₂C(═CH₂)C(O)OH, or a compound of formula (III) wherein:
   L² represents CH₂ monoalkoxylate or CH₂ polyalkoxylate,
   R² represents Q³OQ⁴OC(O)C(CH₃)═CH₂ or Q³OQ⁴OC(O)C(H)═CH₂,
   Q³ represents a divalent residue of an asymmetric diisocyanate compound, preferably chosen among tolyl-1,3-diisocyanate (TDI) and isophorone diisocyanate (IPDI),
   Q⁴ represents CH₂, CH₂—CH₂, CH₂ monoalkoxylate, CH₂—CH₂ monoalkoxylate, CH₂ polyalkoxylate or CH₂—CH₂ polyalkoxylate;
a compound of formula (IV):

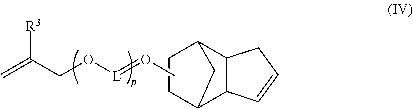

wherein:
   R³ independently represents H or CH₃,
   L³ independently represents a straight or branched C₁-C₂₀-alkylene group and
   n independently represents 0 or an integer ranging from 1 to 30, for example from 1 to 20, particularly from 1 to 15, in particular from 1 to 10;
a compound chosen among the di(meth)acrylates such as polyalkylene glycol di(meth)acrylate, in particular polypropylene glycol di(meth)acrylate, ethylene glycol di(meth)acrylate, polyethylene glycol di(meth)acrylate, triethylene glycol di(meth)acrylate, 1,3-butylene glycol di(meth)acrylate, 1,6-butylene glycol di(meth)acrylate, 1,6-hexanediol di(meth)acrylate, neopentyl glycol di(meth)acrylate, 1,9-nonanediol di(meth)acrylate, but also 2,2′-bis(4-(acryloxy-propyloxyphenyl)propane, 2,2′-bis(4-(acryloxydiethoxy-phenyl)propane; the tri(meth)acrylate compounds such as trimethylolpropane tri(meth)acrylate and ethoxylated trimethylolpropane tri(meth)acrylate, trimethylolethane tri(meth)acrylate, pentaerythritol tri(meth)acrylate and tetramethylolmethane tri(meth)acrylate; the tetra(meth)acrylate compounds such as di-trimethylolpropane tetra(meth)acrylate, tetramethylolmethane tetra(meth)acrylate and pentaerythritol tetra(meth)acrylate; the hexa(meth)acrylate compounds such as dipentaerythritol hexa(meth)acrylate; the penta(meth)acrylate compounds such as dipentaerythritol penta(meth)acrylate; the allyl compounds such as allyl (meth)acrylate, diallyl phthalate, diallyl itaconate, diallyl fumarate, diallyl maleate; the polyallyl sucrose ethers with from 2 to 8 groups per molecule, the pentaerythritol polyallyl ethers such as pentaerythritol diallyl ether, pentaerythritol triallyl ether and pentaerythritol tetraallyl ether; the trimethylolpropane polyallyl ethers such as trimethylolpropane diallyl ether and trimethylolpropane triallyl ether;
a compound chosen among the polyunsaturated compounds in particular divinyl glycol, divinyl benzene, divinylcyclohexyl and methylenebisacrylamide;
a compound chosen among the trifunctional cross-linking monomers, in particular trimethylolpropane tri(meth)acrylate (TMPTA), ethoxylated trimethylolpropane tri(meth)acrylate such as for example TMPTA 3EO;
a compound chosen among ethylene glycol di(meth)acrylate, methylenebisacrylamide, diallylphthalate, diallylmaleate;
a mixture of two different monomers, for example EGDCPEA (ethylene glycol dicyclopentenyl ether acrylate) and TMPTA or else EGDCPEA and TMPTA 3EO;
a compound of formula (V):

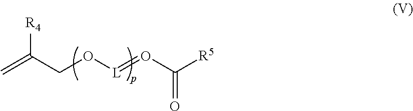

wherein:
   R⁴ independently represents H or CH₃,
   R⁵ independently represents —C(H)═CH₂, —C(CH₃)═CH₂, —C(H)═C(H)C(O)OH, —C(H)═C(H)CH₃, —C(═CH₂)CH₂C(O)OH, —CH₂C(═CH₂)C(O)OH,
   L⁴ independently represents an ethylene, propylene or butylene group and
   p independently represents 0 or an integer or decimal ranging from 1 to 30, preferably p represents an integer or decimal ranging from 1 to 18, from 1 to 15 or from 2 to 16 or else from 2 to 12; more preferentially a compound chosen among a compound (d1) of formula (V) wherein R⁴ represents H, R⁵ represents —C(H)═CH₂, L⁴ represents CH₂—CH₂ and p represents 10 (CAS number 99742-80-0); also particularly preferably a compound (d2) of formula (V) wherein R⁴ represents H, R⁵ represents —C(CH₃)═CH₂, L⁴ represents CH₂—CH₂ and p represents 3,5 (CAS number 121826-50-4); also particularly preferably a compound (d3) of formula (V) wherein R⁴ represents H, L⁴ represents CH₂—CH₂, R⁵ represents —C(CH₃)═CH₂ and p represents 10 (CAS number 121826-50-4).

When preparing copolymer P, the amounts of the compounds used may vary. Preferably according to the invention, copolymer P comprises:
from 25 to 60% by weight of monomer (a),
from 39.89 to 59% by weight of monomer (b),
from 0.1 to 8% by weight of monomer (c) and
from 0.01 to 8% by weight of monomer (d).

Also preferably according to the invention, copolymer P comprises:
from 25 to 60% by weight of monomer (a),
from 44.89 to 59% by weight of monomer (b),
from 0.1 to 8% by weight of monomer (c) and
from 0.01 to 3% by weight of monomer (d).

Also preferably according to the invention, copolymer P comprises:
from 30 to 50% by weight of monomer (a),
from 49.89 to 54% by weight of monomer (b),
from 0.1 to 8% by weight of monomer (c) and
from 0.01 to 8% by weight of monomer (d).

Also preferably according to the invention, copolymer P comprises:
from 25 to 45% by weight of monomer (a),
from 54.89 to 59% by weight of monomer (b),
from 0.1 to 8% by weight of monomer (c) and
from 0.01 to 8% by weight of monomer (d).

More preferably according to the invention, copolymer P comprises from 39.89 to 59% by weight of monomer (b) relative to the amount by weight of monomers (a), (c) and (d). According to the invention, copolymer P is different from a copolymer prepared without methacrylic acid but from 30.0% by weight of acrylic acid, from 58.2% by weight of ethyl acrylate, from 7.5% by weight of 2,2,2-trifluoroethyl methacrylate, from 2.5% by weight of acrylamido-2-methylpropane sulphonic acid and from 0.8% by weight of ethylene dimethacrylate.

Copolymer P according to the invention has many particularly advantageous properties. In particular, copolymer P has a particular glass transition temperature (Tg). Preferably, copolymer P according to the invention has a glass transition temperature (Tg), calculated using the Flory-Fox equation, of less than 60° C., preferably less than 30° C. The Flory-Fox equation makes it possible to calculate the glass transition temperature of the copolymer from the parameters of the monomers used for its preparation, with the exception of any monomers (e) used.

Also preferably, copolymer P according to the invention is fully or partially neutralised, preferably using a compound chosen among NaOH, KOH, LiOH, ammonium derivatives, ammonia, amine bases, for example triethanolamine, aminomethyl propanol or 2-amino-2-methyl-propanol (AMP) and combinations thereof.

In addition to compounds a, b, c and d, copolymer P according to the invention can be prepared by a polymerisation reaction that also uses at least one hydrophobic monomer (e), in particular a hydrophobic associative monomer (e). Preferably according to the invention, the monomer (e) comprises a polymerisable olefinic unsaturation, polyalkylene glycol groups and a hydrophobic end group. Preferably according to the invention, the hydrophobic end group is a straight, branched or cyclic, saturated, unsaturated or aromatic hydrocarbon group comprising from 6 to 40 carbon atoms. Preferably according to the invention, monomer (e) is a compound of formula (VI):

R⁶-(EO)_{q}-(PO)ᵣ-R⁷ (VI)
wherein:
   q and r, identical or different, independently represent 0 or an integer or decimal, less than 150, the sum q+r ranging from 5 to 150, preferably the sum q+r ranging from 10 to 150, advantageously from 10 to 100, more advantageously from 10 to 60,
   EO represents a CH₂CH₂O group,
   PO independently represents a group chosen among CH(CH₃)CH₂O and CH₂CH(CH₃)O,
   R⁶ represents a group comprising at least one polymerisable olefinic unsaturation, preferably a group chosen among acrylate, methacrylate, acryl urethane, methacryl urethane, vinyl, allyl, methallyl, isoprenyl, an unsaturated urethane group, in particular acryl urethane, methacryl urethane, α-α′-dimethyl-isopropenyl-benzyl urethane, allyl urethane, more preferentially a group chosen among acrylate, methacrylate, acryl urethane, methacryl urethane, vinyl, allyl, methallyl and isoprenyl, even more preferentially a methacrylate group,
   R⁷ independently represents a straight, branched or cyclic, saturated, unsaturated or aromatic hydrocarbon group comprising from 6 to 40 carbon atoms, preferably a hydrocarbon group comprising from 6 to 32 carbon atoms, more preferentially from 8 to 30 carbon atoms.

Preferentially, R⁷ represents an alkyl group derived from a Guerbet alcohol of formula (VII):

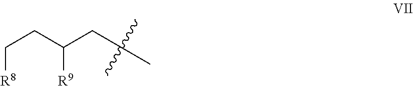

wherein R⁸ and R⁹ independently represent a C₆-C₄₀-alkyl group, preferably a C₆-C₃₂-alkyl group.

Also preferably according to the invention, R⁷ represents a straight alkyl or alkenyl group, comprising from 6 to 40 carbon atoms, in particular a cyclohexyl group. According to the invention, R⁷ may also represent an alkyl group derived from an alcohol obtained by an oxo reaction.

According to the invention, R⁷ may represent an aromatic group comprising from 6 to 40 carbon atoms, preferentially from 6 to 32 carbon atoms, more preferentially from 6 to 30 carbon atoms.

According to the invention, R⁷ may represent a group of formula (VIII):

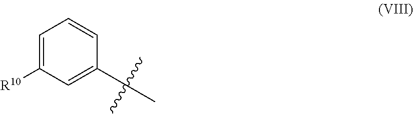

wherein R¹⁰ represents a hydrocarbon group of formula C₁₅H₃₁₋₈ wherein s represents 0, 2, 4 or 6; R¹⁰ may thus comprise 0, 1, 2 or 3 ethylenic unsaturations (double bond). Such a group of formula (VIII) is advantageously derived from cardanol, and is therefore of bio-sourced origin.

According to the invention, R⁷ may represent a group comprising from 2 to 5 phenyl groups, such as a tristyrylphenyl (TSP) group of formula (IX):

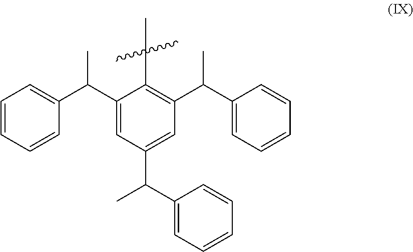 [SMILES: *c1c(C(C)c2ccccc2)cc(C(C)c2ccccc2)cc1C(C)c1ccccc1]

or a distyrylphenyl group (DSP) of formula (X) or of formula (XI):

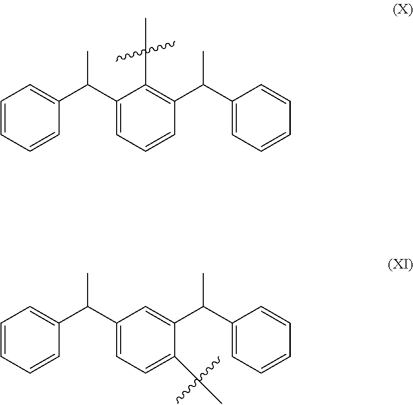 [SMILES: *c1c(C(C)c2ccccc2)cccc1C(C)c1ccccc1 ; *c1ccc(C(C)c2ccccc2)cc1C(C)c1ccccc1]

or a pentastyrylcumylphenyl group.

In particular, R⁷ independently represents a straight, branched or cyclical, saturated, unsaturated or aromatic hydrocarbon group comprising from 6 to 40 carbon atoms, preferably a straight or branched C₆-C₄₀-alkyl group, preferably a straight or branched C₈-C₃₀ alkyl group, a C₆-C₄₀-aryl group, preferably a C₈-C₃₀-aryl group, preferentially comprising 2 to 5 phenyl groups, for example a tristyrylphenyl group.

Preferably according to the invention, q represents an integer or decimal, advantageously an integer, greater than or equal to 10.

Preferably according to the invention, the q value is strictly greater than the r value. More preferably according to the invention, the respective amounts by weight of q and r range from (q=100 and r=0) to (q=70 and r=30).

According to one variant, r is null and q represents an integer or decimal, advantageously a number ranging from 10 to 100, advantageously ranging from 10 to 60, more advantageously ranging from 20 to 60, even more advantageously ranging from 20 to 40.

According to another variant, each of r and q is different from 0. In particular, q and r, identical or different, independently represent an integer or decimal, advantageously a number ranging from 5 to 100, the sum q+r varying from 10 to 150, advantageously from 10 to 100, more advantageously from 10 to 60. Preferably according to the invention, the q value is strictly greater than the r value. In this case and preferably according to the invention, the respective amounts by weight of q and r range from (q=90 and r=10) to (q=70 and r=30).

More preferably according to the invention, n represents 0.

Advantageously according to the invention, copolymer P can comprise from 0.4 to 30% by weight of monomer (e) relative to the total amount of monomers. Copolymer P according to the invention can be prepared according to methods known as such. Specifically, copolymer P according to the invention is prepared by a polymerisation reaction using the different compounds a, b, c and d, optionally compound e, by a radical polymerisation reaction, for example a polymerisation reaction in emulsion, in dispersion or in solution. Advantageously, copolymer P is prepared in water, preferably in the presence of at least one surfactant compound, for example sodium dodecyl sulphate or sodium dodecyl laurate.

The preparation of copolymer P according to the invention can also use one or more compounds, in particular at least one initiator compound, alone or in combination with at least one chain transfer agent. As examples of initiator compounds, one compound can be used that is chosen among the azoic initiator compounds (for example azobisisobutyronitrile), a peroxide compound, preferably hydrogen peroxide, benzoyl peroxide, benzoyl hydroperoxide and mixtures thereof. Alkaline metal persulphates can also be mentioned, particularly sodium persulphate and potassium persulphate, ammonium persulphate, partially water-soluble peroxides, particularly succinic peracid, t-butyl hydroperoxide, cumyl hydroperoxide, persulphates combined with a copper ion, a ferrous ion, a sulphite ion or a bisulphite ion and mixtures thereof. As examples of chain transfer agents, mercaptan compounds can be used, in particular mercaptan compounds comprising at least 4 carbon atoms such as butylmercaptan, n-octylmercaptan, n-dodecylmercaptan, tert-dodecylmercaptan, isooctyl 3-mercaptopropionate.

According to the invention, the radical-initiating or radical-generating compound can therefore be combined with at least one controlled radical polymerisation transfer agent, in particular a RAFT (reversible addition-fragmentation chain transfer) transfer agent.

Preferably, the reaction is a radical polymerisation reaction in emulsion.

Thus, preferably according to the invention, the preparation in water of copolymer P makes it possible to obtain a polymeric aqueous composition in the form of an emulsion.

The particularly advantageous properties of copolymer P according to the invention make it possible to use it in many technical fields. In particular, copolymer P according to the invention can be used to improve the viscoelastic properties and the suspensive properties of a composition, in particular of an aqueous composition, preferably of an aqueous composition comprising particles. Thus, in addition to copolymer P, the invention provides an aqueous composition C comprising at least one copolymer P according to the invention. Preferably, the aqueous composition C according to the invention comprises at least one copolymer P according to the invention and solid, liquid or gaseous particles, and optionally a binder compound.

Preferably, composition C according to the invention comprises from 0.1 to 5% by weight of copolymer P. More preferably, composition C according to the invention comprises from 0.5 to 3% by weight of copolymer P.

Preferably for composition C according to the invention, the particles are particles of a product chosen among a cosmetic product, a phytosanitary product, a fertilizer, a coating product. More preferably for composition C according to the invention, aqueous composition C is a varnish composition comprising a copolymer P, particles of a pigment and a binder compound in the form of latex, optionally a pigment-dispersing compound.

Preferably according to the invention, the binder compound has a glass transition temperature, calculated using the Flory-Fox equation, which is the same +/−10° C. as the glass transition temperature of copolymer P. Also preferably according to the invention, the binder compound has a glass transition temperature, calculated using the Flory-Fox equation, which is the same +/−5° C. as the glass transition temperature of copolymer P. According to the invention, the preferred binder compound is an acrylic compound, an alkyd compound or an alkyd-urethane compound or else a styrene-acrylic compound or a styrene-butadiene compound.

The properties of copolymer P according to the invention make it possible to use it under conditions that can vary significantly. In particular, the properties of copolymer P can be used at variable pH values. Preferably, aqueous composition C according to the invention has a pH ranging from 3 to 13, preferably a pH ranging from 5 to 13. Also preferably, the pH of aqueous composition C ranges from 4 to 8 or from 5 to 7.

The invention also relates to a method for preparing a viscoelastic and suspensive aqueous composition C, comprising the introduction of at least one copolymer P according to the invention and the stirring of composition C. Copolymer P makes it possible to improve the properties of composition C according to the invention. In particular, copolymer P makes it possible to improve the stability of this composition, especially for a varnish composition C. Thus, the invention also relates to a method for stabilising a varnish composition C comprising the introduction into an aqueous varnish base composition C of at least one copolymer P according to the invention. Preferably, the varnish base composition C comprises a binder compound in the form of a latex with a glass transition temperature that is the same +/−10° C. as the glass transition temperature of copolymer P, calculated using the Flory-Fox equation. Also preferably for this method, the binder compound has a glass transition temperature, calculated using the Flory-Fox equation, which is the same +/−5° C. as the glass transition temperature of copolymer P.

Copolymer P according to the invention has particularly advantageous properties for controlling various components of the rheology of an aqueous composition. In particular, copolymer P according to the invention makes it possible to control the flow threshold of an aqueous composition.

The flow threshold corresponds to the value of the shear stress applied to an aqueous composition that makes this composition flow. In the absence of sufficient stress, the viscosity of this aqueous composition prevents its spontaneous flow at an acceptable time scale.

Thus, the invention also provides a method for controlling the flow threshold of an aqueous composition comprising the introduction into the aqueous composition of at least one copolymer P according to the invention. Preferably, the flow threshold of this aqueous composition, measured according to the method described in the examples, is greater than 0.1 Pa, more preferentially greater than 0.5 Pa or greater than 2 Pa, preferably greater than 4 Pa.

More preferably, composition C according to the invention comprises from 0.1 to 5% by weight of copolymer P and has a flow threshold greater than 0.1 Pa, more preferentially greater than 0.5 Pa, or greater than 2 Pa, preferably greater than 4 Pa. Also more preferably, composition C according to the invention comprises from 0.5 to 3% by weight of copolymer P and has a flow threshold greater than 0.1 Pa, more preferentially greater than 0.5 Pa, or greater than 2 Pa, preferably greater than 4 Pa.

According to the invention, the particular, advantageous or preferred characteristics of copolymer P according to the invention define compositions according to the invention and methods according to the invention which are also particular, advantageous or preferred compositions and methods according to the invention.

The following examples illustrate the various aspects of the invention.

### EXAMPLES

### Example 1: Preparation and Characterisation of Copolymers According to the Invention

The preparation reactions of copolymers P according to the invention were carried out in a cylindrical glass reactor with a usable volume of 1 litre equipped with mechanical anchor stirring and an oil bath heating. Stirring is maintained throughout the preparation.

The following monomers were used:
monomer a:
   compound (a1), acrylic acid (AA),
   compound (a2), methacrylic acid (MAA),
monomer b: compound (b1), ethyl acrylate (EA),
compound c: compound (c1), 2-acrylamido-2-methylpropane sulphonic acid (AMPS) sodium salt,
cross-linking compound d:
   compound (d1), ethylene glycol dimethacrylate (EDMA),
   compound (d2), diallyl phthalate (DAP),
   compound (d3), compound of formula IV wherein R³ represents H, L³ represents a CH₂CH₂ group and n represents 1,
   compound (d4), triallyl pentaerythritol (APE),
   compound (d5), trimethylolpropane triacrylate (TMPTA),
hydrophobic monomer e: compound (el) of formula VI wherein R⁶ represents a methacrylate group, q represents 25, EO represents a CH₂CH₂O group, r represents 0 and R⁷ represents a group of formula VII wherein R⁸ represents a straight C₆-alkyl group and R⁹ represents a straight C₁₀-alkyl group.

Preparation and Characterisation of Copolymer (P1) According to the Invention

In the reactor, 460 g of deionised water and 6.48 g of sodium dodecyl sulphate are introduced. In a first glass beaker and according to the proportions shown in Table 1, 102.14 g of monomer (a1), 160.20 g of monomer (b1), 4.57 g of compound (d1), 20.05 g of monomer (el), 3.38 g of sodium dodecyl sulphate and 124 g of deionised water are weighed. In a second glass beaker, 0.914 g of ammonium persulphate is weighed and then dissolved in 10 g of deionised water. In a third glass beaker, 0.100 g of sodium metabisulphite is weighed and then dissolved in 10 g of deionised water. In a fourth container, such as a disposable syringe, 5.27 g of compound (c1) at 50% by weight in water are weighed.

The reactor content is heated to 76° C.±2° C.

The reagents from the 4 containers are introduced into the polymerisation reactor in 2 hours and 30 minutes at a temperature of 76° C.±2° C. The pumps are rinsed with deionised water. Then, 0.3 g of ammonium persulphate dissolved in 20 g of deionised water are introduced into the reactor in 1 hour.

Then, bake for 1 hour before allowing the medium to cool and then filtering it.

A copolymer (P1) at 30.6% by weight of solids content (SC, measured with a microwave scale) is obtained. Its glass transition temperature (Tg) is calculated using the Flory-Fox equation. The composition and characteristics of copolymer (P1) are shown in Table 1.

Preparation and Characterisation of Copolymers (P2) to P(14) According to the Invention

These polymers are prepared in a similar way to copolymer (P1). Their compositions and characteristics are shown in Table 1.

TABLE 1

| | Monomer (amount - % by weight) | | | | | SC | Tg |
|---|---|---|---|---|---|---|---|
| Copolymer | (a1) | (b1) | (c1) | (d) | (e) | (%) | (° C.) |
| P1 | 35.27 | 55.32 | 0.90 | 1.59 (d1) | 6.92 (e1) | 30.6 | 43 |
| P2 | 38.00 | 60.84 | 0.90 | 0.26 (d2) | / | 26.9 | 16 |
| P3 | 33.98 | 64.72 | 1.10 | 0.20 (d3) | / | 30.7 | 12 |
| P4 | 31.54 | 67.31 | 0.60 | 0.55 (d1) | / | 29.5 | 9 |
| P5 | 41.16 | 57.98 | 0.60 | 0.25 (d2) | / | 27.4 | 18 |
| P6 | 36.33 | 53.84 | 0.89 | 1.54 (d1) | 7.41 (e1) | 28.4 | 46 |
| P7 | 36.56 | 62.24 | 0.90 | 0.30 (d2) | / | 29.5 | 14 |
| P8 | 33.98 | 64.72 | 0.70 | 0.60 (d3) | / | 27.8 | 12 |
| P9 | 36.63 | 62.36 | 0.90 | 0.11 (d4) | / | 29.9 | 13 |
| P10 | 34.98 | 63.79 | 1.08 | 0.14 (d4) | / | 29.4 | 13 |
| P11 | 27.71 | 71.11 | 1.09 | 0.09 (d4) | / | 29.2 | 5 |
| P12 | 36.61 | 62.32 | 0.90 | 0.17 (d4) | / | 29.6 | 14 |
| P13 | 35.00 | 63.82 | 1.09 | 0.09 (d5) | / | 29.8 | 12 |
| P14 | 27.72 | 71.13 | 1.09 | 0.06 (d4) | / | 29.0 | 5 |

### Example 2: Preparation and Characterisation of the Properties of Aqueous Compositions C₁ to C₃ According to the Invention

Copolymer P1 prepared according to example 1 is mixed with deionised water in an amount of 1% by dry weight and then an aqueous sodium hydroxide solution (50% by weight) is added to fully neutralise the composition. Aqueous composition C₁ according to the invention is obtained. Aqueous compositions C₂ and C₃ according to the invention are similarly prepared from copolymers P2 and P3 according to the invention.

The Brookfield viscosity of these compositions is then measured at 25° C. and 100 rpm using a Brookfield DV1 viscometer equipped with a spindle adapted to the viscosity range of the composition. The flow threshold of these compositions, which is the stress applied that results in the flow of the aqueous composition, is also measured. A stress ramp varying from 0.01 to 1,000 Pa is used for a period of 3,000 seconds with a balancing time of 30 seconds between each measurement. The flow threshold stress was measured at 25° C. using a Haake Mars III imposed stress rheometer (ThermoFisher Scientific) with CP60-1/S cone-plane geometry (60 mm diameter, 1° truncation angle) and then processed using RheoWin Data Manager software. The results obtained are shown in Table 2.

TABLE 2

| Aqueous | Copolymer | Brookfield Viscosity | Flow threshold |
|---|---|---|---|
| composition | used | (mPa · s) | (Pa) |
| C1 | P1 | 1,800 | 19.4 |
| C2 | P2 | 170 | 0.68 |
| C3 | P3 | 390 | 0.65 |

The viscoelasticity of aqueous compositions C₁, C₂ and C₃ according to the invention is also determined based on the frequency with low strains. The imposed strain is sinusoidal in the shape γ=γ₀ sin(ωt) with γ₀ set at 1%. The frequency ω follows a logarithmic variation from high to low frequencies (from 100 to 0.01 Hz). The measured stress response is of type σ=σ₀ sin(ωt+δ) with δ being the phase offset. If necessary, the stress signal can also be broken down into an in-phase part (solid response) and the opposite phase part (liquid response). These two contributions are expressed in the formula σ=γ0G′ sin(ωt)+γ0G″cos(ωt) wherein G′ represents the storage modulus (or elastic modulus) and G″ represents the loss modulus (or viscous modulus).

The solid or liquid nature of the aqueous composition sample is thus determined. When G′ >G″, then the sample is solid. When G″>G′, then the sample is liquid. We can define tan δ=G″/G′ which is the loss angle. If tan δ=0, the behaviour is solid-elastic, if tan δ=1, the behaviour is viscous. When 0<tan δ<1, the composition being assessed has a viscoelastic behaviour. The results obtained are shown in Table 3.

TABLE 3

| Aqueous | Copolymer | Tangent δ | | | |
|---|---|---|---|---|---|
| composition | used | at 0.01 Hz | at 0.1 Hz | at 1 Hz | at 10 Hz |
| C1 | P1 | 0.48 | 0.51 | 0.58 | 0.41 |
| C2 | P2 | 0.29 | 0.26 | 0.33 | 0.58 |
| C3 | P3 | 0.21 | 0.22 | 0.31 | 0.60 |

The copolymers according to the invention therefore make it possible to thicken aqueous compositions considerably. The aqueous compositions obtained have a high flow threshold. In addition, the tangent 6 value of these aqueous compositions according to the invention is systematically between 0 and 1 thus demonstrating their viscoelastic character. These properties are obtained in the absence of methacrylic acid during the preparation of the copolymers according to the invention.

1. A copolymer, prepared in the absence of methacrylic acid, by at least one polymerization of components comprising:
(a) an anionic monomer comprising acrylic acid and/or an acrylic acid oligomer, optionally in salt form;
(b) a non-ionic monomer comprising styrene, a C1-C8 acrylate ester, and/or a C1-C8 methacrylate ester;
(c) compound comprising 2-acrylamido-2-methylpropane sulfonic acid, 2-sulfoethyl methacrylate, sodium methallyl sulfonates, and/or styrene sulfonate, optionally in salt form; and
(d) a cross-linking compound or second compound comprising a first and a second polymerizable olefinic unsaturation.

2. The copolymer of claim 1, wherein the anionic monomer (a) comprises acrylic acid or a mixture of acrylic acid oligomers of formula (I): 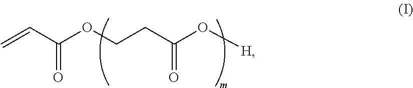
wherein m is in a range of from 1 to 10.

3. The copolymer of claim 1, wherein the non-ionic monomer (b) comprises:
an alkyl acrylate,
an alkyl methacrylate,
an aryl acrylate, and/or
an aryl methacrylate.

4. The copolymer of claim 1, wherein the compound (c) comprises 2-acrylamido-2-methylpropane sulfonic acid, optionally as a sodium and/or ammonium salt.

5. The copolymer of claim 1, wherein the second compound (d) is present and comprise a polyunsaturated aromatic monomers.

6. The copolymer of claim 1, comprising:
the anionic monomer (a) in a range of from 25 to 60 wt %;
the non-ionic monomer (b) in a range of from 39.89 to 59 wt. %;
the compound (c) in a range of from 0.1 to 8 wt. %; and
the cross-linking compound or second compound (d) in a range of from 0.1 to 8 wt. %.

7. The copolymer of claim 1, having a glass transition temperature, calculated using the Flory-Fox equation, of less than 60° C.

8. The copolymer of claim 1, which is at least partially neutralized.

9. The copolymer of claim 1, wherein the components further comprise a hydrophobic monomer (e) comprising a polymerizable olefinic unsaturation, polyalkylene glycol groups, and a hydrophobic end group.

10. The copolymer of claim 9, wherein the hydrophobic monomer (e) is of formula (VI):
R⁶-(EO)_{q}-(PO)ᵣR⁷ (VI),
wherein
q and r are independently in a range of from 0 to 150, a sum q+r being in a range of from 5 to 150,
EO is a CH₂CH₂O group,
PO is independently CH(CH₃)CH₂O or CH₂CH(CH₃)O,
R⁶ is a group comprising a polymerizable, olefinic unsaturation,
R⁶ is independently a aromatic hydrocarbon group comprising from 6 to 40 carbon atoms.

11. The copolymer of claim 9, comprising the hydrophobic monomer (e) in a range of from 0.4 to 30 wt. % relative to total monomer weight.

12. An aqueous composition, comprising:
the copolymer of claim 1;
particles; and
optionally, a binding compound.

13. The composition of claim 12, comprising the copolymer in a range of from 0.1 to 5 wt. %.

14. The composition of claim 12, wherein the particles are particles of a cosmetic product, a phytosanitary product, a fertilizer, or a coating product.

15. The composition of claim 12, which is an aqueous varnish composition comprising the copolymer, the particles of a pigment, a latex binder compound as the binding compound, and optionally a pigment-dispersing compound.

16. The composition of claim 12, having a pH in a range of from 3 to 13.

17. A method for preparing an aqueous composition with one or more viscoelastic and suspensive properties, the method comprising:
introducing at least one of the copolymer of claim 1, particles, and, optionally, a binding compound, to form the composition; and
stirring the composition.

18. A method for stabilizing a varnish composition, the method comprising:
introducing into an aqueous varnish base composition at least one of the copolymer of claim 1.

19. A method for controlling the flow threshold of an aqueous composition, the method comprising:
introducing into the aqueous composition at least one of the copolymer of claim 1.

20. A copolymer, comprising, in polymerized form:
(a) an anionic monomer comprising acrylic acid and/or an acrylic acid oligomer, optionally in salt form;
(b) a non-ionic monomer comprising styrene, a C1-C8 acrylate ester, and/or a C1-C8 methacrylate ester;
(c) a compound comprising 2-acrylamido-2-methylpropane sulfonic acid, 2-sulfoethyl methacrylate, sodium methallyl sulfonate, and/or styrene sulfonate, optionally in salt form; and
(d) a cross-linking compound or compound comprising a first and a second polymerizable olefinic unsaturation,
wherein the copolymer comprises no methacrylic acid.
